(19) United States
(12) Patent Application Publication
(10) Pub. No.: US 2020/0054702 A1
(43) Pub. Date: Feb. 20, 2020

(54) Methodology and Formulation for Creating a Powder of an Encapsulated Cannabis-Based Component Embedded in a Polymer Matrix

(71) Applicants: Michael HELLER, Broadview Heights, OH (US); MJ WOOLY CORPORATION, Broadview Heights, OH (US)

(72) Inventor: Michael HELLER, Broadview Heights, OH (US)

(21) Appl. No.: 16/610,185

(22) PCT Filed: May 1, 2018
(86) PCT No.: PCT/US2018/030396

§ 371 Date: Nov. 1, 2019

Related U.S. Application Data
(60) Provisional application No. 62/492,442, filed on May 1, 2017.

(51) Int. Cl. A61K 36/185; A61K 31/05; A61K 31/352; A61K 9/16

(52) U.S. Cl. CPC A61K 36/185; A61K 31/05; A61K 9/1682; A61K 9/1629; A61K 31/352

(57) ABSTRACT

Provided are compositions and methods of forming a particulate material derived from a cannabis plant. The method includes introducing a component including at least one of: (i) a cannabinoid, and (ii) a terpene, to a polymer to produce a polymeric mixture. The component is dispersed in the polymeric mixture, which is then at least partially dehydrated to encapsulate the component within a polymeric material derived from the polymer. The polymeric material is water soluble. The dehydrated polymeric mixture is processed to form particulates comprising the component encapsulated within shells formed from the polymeric material.

### CROSS-REFERENCE TO RELATED APPLICATIONS

This disclosure is a 371 of International Application PCT/US2018/030396 serial number, filed May 1, 2018, entitled “Methodology and Formulation for Creating Powder of an Encapsulated Cannabis-Based Component Embedded in a Polymer Matrix,” inventor Michael HELLER, which claims benefit of U.S. Provisional Application 62/492,442 serial number, filed May 1, 2017 entitled “Methodology and Formulation for Creating a Powder of Nano-Encapsulated Cannabinoids Embedded in a Polymer Matrix,” inventor Michael HELLER, and assigned to the present assignee, which is incorporated herein in its entirety by reference.

### BACKGROUND OF THE INVENTION

### Field of the Invention

This application relates generally to a composition and method for forming a composition of a powdered cannabinoid, terpene, essential oil, and the like, with self-emulsifying properties.

### Description of Related Art

Conventional cannabinoid compositions have traditionally exhibited limited shelf life. Further, when added to a liquid to form a solution for ingestion, conventional cannabinoids often require the use of separate, complex emulsifying agents and involved processing techniques to form a desirable product. And, some compositions such as terpenes and essential oils, for example, may include one or more volatile components. Processes that utilize highly-elevated temperatures can cause at least a portion of the volatile components to be flashed from solution and excluded from the end product, or undergo an undesirable change to their chemical structure.

### BRIEF SUMMARY OF THE INVENTION

According to one aspect, the subject application involves a composition and a method of making a composition for powdered cannabinoids, terpene, essential oils, and the like, having a self-emulsifying property. For example, encapsulated cannabinoid emulsions can be cast or embedded in an aqueous polymeric solution or other polymeric solution. The water, for example, can be removed from the combined substances, resulting in the encapsulated cannabinoids being embedded in the polymer agglomeration or matrix. The encapsulated cannabinoids are substantially homogeneously dispersed and entrapped in the polymer network. In this fashion, encapsulation of cannabinoids can be complete, and particle sizes substantially maintained in a range up to twenty (20 μm) microns. The polymer matrix can be dehydrated and then milled into a free flowing powder. As a powder, the encapsulated cannabinoids are protected from environmental and chemical degradation for extended periods of time, such as several months to 2, 3 or more years. The resulting powder can be rapidly dissolved, optionally in less than 5 min when submerged or otherwise exposed to an aqueous environment, for example. Once dissolved the polymers dissociate from the encapsulated cannabinoids and allow them to quickly and evenly disperse into solution. Once the powder of encapsulated cannabinoids is completely dissolved it forms a shelf stable emulsion that has ideal cannabinoid particle sizes for efficient absorption in the body. Embodiments of the ideally-encapsulated cannabinoids have particle sizes between about fifty (50 nm) nanometers and about twenty (20 μm) microns. The resulting emulsion observes a reduction in the time for the first onset of action in the body of a consumer who ingests the emulsion, optionally as an additive to a consumable composition, attributed to the encapsulated cannabinoid, terpene, essential oil, etc.

For example, a consumer who ingests the cannabinoid, terpene, essential oil, etc., that was formerly encapsulated and dissolved as described herein may experience the onset of an effect of a cannabinoid acting on cannabinoid receptors in cells that alter neurotransmitter release in the brain within 30 min of ingestion. Such an onset time is approximately half of the time required to experience the onset of the cannabinoid effect as a result of ingestion of a traditional cannabinoid that was not encapsulated in a polymeric powder shell that has been dissolved as described herein. The result is a substantially higher bioavailability (e.g., 2-5 fold) compared to a traditional cannabinoid dissolved in an edible oil.

According to another aspect, the subject application involves a method of forming a particulate material derived from a cannabis plant. The method includes introducing a component comprising at least one of: (i) a cannabinoid, and (ii) a terpene, to a polymer to produce a polymeric mixture. The component is dispersed in the polymeric mixture, and the polymer mixture with the dispersed component is at least partially dehydrated to encapsulate the component within a polymeric material derived from the polymeric mixture. The polymeric material can be water soluble. The dehydrated polymeric mixture, which is solidified, is then processed to form particulates comprising the component encapsulated within shells formed from the polymeric material.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

The present disclosure is directed generally to a composition of a powdered cannabinoid, terpene, essential oil, or other such substance, having a self-emulsifying property, and a method of making such a powder. Although the present disclosure is not limited to powdered cannabinoids, the embodiment of a cannabinoid is described primarily below for the sake of brevity, and to clearly describe the present methods.

The present disclosure involves a cannabinoid formulation that comprises encapsulated cannabinoids entrapped in a polymer matrix. The cannabinoid embedded polymer matrix can be in a gel or colloidal solution (e.g., “sol”) state or can be dehydrated to make a millable free flowing powder. The polymer matrix can be readily dissolvable in aqueous environments upon which the encapsulated cannabinoids can be released into the aqueous medium creating a substantially homogenous and substantially-perpetual stable emulsion.

The Component or Active Ingredient

The component, interchangeably referred to herein as the active ingredient, can include oils, resins and molecules derived from the cannabis plant or modeled after the components found in the cannabis plant. This includes cannabinoids and terpenes that are natural, semi-natural, synthetic or combinations thereof. The active ingredients are molecules that engage the endocannabinoid system, elicit a physiological response or support/enhance a physiological response. These active ingredients include cannabinoids such as: delta-9-tetrahydrocannabinolic acid (THCa), delta-9-tetrahydrocannabinol (THC), cannabidiol acid (CBDa), cannabidiol (CBD), cannabinol (CBN), cannabigerol (CBG), cannabichromene (CBC), tetrahydrocannabivarin (THCV), cannabidivarin (CBDV), or combinations thereof. The addition of terpenes can be used to enhance or alter the effect of cannabinoids, or elicit their own physiological reaction. Terpenes found in the cannabis plant include: alpha-pinene, linalool, myrcene, limonene, ocimene, terpinolene, terpineol, valencene, beta-caryophyllene, geraniol, humulene, phellandrene, carene, terpinene, fenchol, borneol, bisabolol, phytol, camphene, sabinene, camphor, isoborneol, menthol, cedrene, nerolidol, guaiol, isopulegol, geranyl acetate, cymene, eucalyptol, pulegone. Active ingredients are not limited to those mentioned above and can also include: 11-hydroxy-tetrahydrocannabinol, levonantradol, delta-8-tetrahydrocannabinol, nabilone, 3-dimethylneptyllcarboxylic acid homologine, dronabinol. Additionally, cannabinoid analogues or other synthetic and semi-synthetic cannabis based active ingredients can be used as the active pharmaceutical ingredient (API).

The one or more active ingredients to be encapsulated by a polymeric shell can come in a variety of different forms including isolated molecules (isolates), resins, oleo-resins, saps, waxes and oils. The purity of total cannabinoids and/or terpenes can vary from 25% to 100% w/w depending on the separation/isolation techniques used. The following techniques are capable of producing cannabis oils/resins with purities and consistencies acceptable for this invention. These include oils and resins made with primary extractions using: hydrocarbon solvents (butane, propane, hexane, etc.), supercritical and subcritical carbon dioxide, alcohol solvents (C1-C10), rosin pressing (applying heat and pressure), cold pressing, sieve separation (ice water hash, etc.), organic solvents (dichloromethane, dimethylsulfoxide, acetone, ether, naptha, tetrahydrofuran, DMF, etc.), edible solvents (edible oils, propylene glycol, PEG, etc.), molecular distillation, steam distillation, ultrasonic water extraction. Primary extractions typically result in cannabis oils/resins with cannabinoid purities between 25 and 80% w/w. Cannabis oils/resins from primary extractions can be used directly for this invention or be further refined to remove impurities, remove flavors, and improve the consistency of the oil/resin. Secondary purification steps include but are not limited to: distillation (short path, molecular film, thin film), chromatography, precipitation, liquid-liquid extraction and winterization (dewaxing). Following secondary purification cannabis oils/resins reach cannabinoid purities between 50 and 100% w/w.

As specific examples, refined cannabis oils made using either hydrocarbon extraction, super/sub critical carbon dioxide extraction, alcohol extraction, distillation or any other method that results in a high concentration of cannabinoids and terpenes. Embodiments of the cannabis oil purity can include:

25-100% w/w cannabinoids+terpenes

50-100% w/w cannabinoids+terpenes

65-100% w/w cannabinoids+terpenes.

The ratios of cannabinoids and terpenes are dependent on the starting plant material and the extraction/purification techniques used to make the cannabis oils/resins/isolates. It is possible to perform the purification techniques in a way that results in the optimal cannabinoid/terpene ratios. It is also possible to separate the active ingredients into their individual components and then recombine them in the desired ratios. It is further possible to chemically synthesize, derivatize or chemically modify cannabinoids or combinations of cannabinoids to be used as described herein. For example, the cannabinoid/terpene ratio can be:

Pure cannabinoids (e.g., 100% THC, 100% CBD, 100% CBN, 100%, CBG, 100% CBC, 100% THCa, 100% THCV, 100% CBDa, 100% CBDV);

Cannabis oils/resins such as:

60-97% THC (or THCa) with residual cannabinoids and terpenes,

60-97% CBD (or CBDa) with residual cannabinoids and terpenes,

3-97% CBN with residual cannabinoids and terpenes,

3-97% CBG with residual cannabinoids and terpenes,

3-97% CBC with residual cannabinoids and terpenes,

3-97% THCV with residual cannabinoids and terpenes,

3-97% CBDV with residual cannabinoids and terpenes.

The following cannabinoid compounding ratios can be utilized according to various embodiments. However, it is to be understood that the following ratios are only examples, and that any ratios of sufficiently pure cannabinoids and terpenes can be utilized:

Compounding ratios of APIs:

THC/CBD: 0:1-1:0→1:1, 2:1, 3:1, 4:1, 5:1, 10:1, 20:1 etc.,

CBD/THC:0:1-1:0→1:1, 2:1, 3:1, 4:1, 5:1, 10:1, 20:1 etc.,

THC/CBN:0:1-1:0→1:1, 2:1, 3:1, 4:1, 5:1, 10:1, 20:1 etc.,

CBN/THC: 0:1-1:0→1:1, 2:1, 3:1, 4:1, 5:1, 10:1, 20:1 etc.,

CBD/CBN:0:1-1:0→1:1, 2:1, 3:1, 4:1, 5:1, 10:1, 20:1 etc.,

CBN/CBD: 0:1-1:0→1:1, 2:1, 3:1, 4:1, 5:1, 10:1, 20:1 etc.,

THC/CBD/CBN: 1-99% THC: 1-99% CBD: 1-99% CBN.

According to alternate embodiments, cannabis oil/resin/isolates devoid of terpenes can be utilized. Terpenes are aroma and flavor molecules that can interfere with the taste of products infused using this invention. Terpenes can be removed from cannabis oils using distillation, chromatography, evaporation/heating, or any methodology that sufficiently purges or removes them from the oil. Sufficient removal of terpenes from the cannabis oils results in flavorless and odorless forms of this invention. Specific embodiments of cannabis oil devoid of terpenes include:

A cannabis oil purified using short path distillation or chromatography,

Terpene content less than 5%, or alternately less than 1%, or alternately less than 0.25%.

According to other embodiments, encapsulated terpenes can be devoid of cannabinoids. In this way cannabinoids can be removed prior to the encapsulation process or non-cannabis derived terpenes can be used. Steam distillation is one example of a process that is capable of isolating terpenes from the cannabis plant or resins from the other components such as the cannabinoids.

Regardless of whether the components described herein are derived from cannabinoids or terpenes, the component can be processed into a liquid or gel state according to the present method, prior to being introduced to, and dispersed in a polymeric mixture. This can promote and ensure even dispersion, and accordingly, complete encapsulation of the component within the polymeric material forming a component of the polymeric mixture. Viscosities for the component or the polymeric mixture of 0.3 centipoise to 250,000 centipoise can be accomplished by heating the material between 15° C. and 100° C. The component should not be exposed to temperatures above 100° C. because above this temperature volatile compounds can begin to evaporate and certain components may chemically degrade or evolve as a result of the elevated temperature.

Carrier

Drugs that have poor aqueous solubility may demonstrate absorption that is dissolution rate-limited, resulting in poor bioavailability and long onset of action times. The bioavailability of water insoluble drugs can optionally be improved by dissolving the drug in a carrier oil or solubilizing agent. This increases the dispersion of the drug relative to the drug not dissolved in a carrier oil or solubilizing agent, resulting in improved absorption in the body. Dispersion and absorption can be even further increased by creating oil-in-water emulsions from the drug solubilized in a carrier oil. A water insoluble drug may have a 2-10 fold increase in bioavailability when made into an oil-in-water emulsion compared to an aqueous suspension of the drug.

The choice of carrier oil (or solubilizing oil/agent) can impact the efficacy of the drug in the body. Different carrier oils affect emulsion systems by impacting the particle size, stability, interfacial barrier partitioning the oil/water phase, emulsion core density. Additionally, different carrier oils affect how water insoluble drugs enter the body by controlling the absorption profile, release profile and by protecting labile drugs from environmental stress factors such as oxidation, hydrolysis and acid/base decomposition.

The first consideration for carrier oil/solvent choice is the solubility of drug in the carrier. For this reason a carrier oil that is capable of significantly solubilizing cannabinoids, terpenes and or essential oils at adequate concentrations is to be chosen. The second consideration for carrier oil/solvent choice is the digestion of the lipid based formulations. The chain length of fatty acids and triglycerides has a significant impact on the digestion of lipids, prior to absorption in body. Shorter chain lipids such as medium chain triglycerides (MCTs) demonstrate fast rates of digestion and help promote further emulsification in the intestine prior to absorption. This may result in a more rapid onset of action in the body. However, the higher rates of digestion may subject active ingredients to enzymatic degradation and lower bioavailability compared to longer chain oils. Longer chain lipids such as long chain triglycerides (LCTs) and vegetable oils take longer to digest but can protect drugs from enzymatic degradation resulting in higher bioavailability.

The third consideration for carrier oil/solvent is the absorption of the drug through the mucosal cells in the mouth and intestine. During digestion triglycerides are broken down into mono/diglycerides and fatty acids which can rapidly and efficiently be absorbed, this facilitates and promotes the absorption of drugs in the body. It is believed that the drug component partitions from the lipid vehicle and/or emulsion before it can be absorbed by the appropriate tissue. Lipids with similar chain lengths to the emulsifiers/surfactants used to create the emulsion may weaken the interfacial barrier and more readily allow for the faster release of drug components.

Carrier Oils:

According to one embodiment, the present compositions and methods can utilize a delivery system that demonstrates a rapid onset of action. In an effort to reduce the time required for absorption in the body, medium chain triglycerides (MCTs) can be chosen as the primary carrier oils. MCTs are substantially, and optionally completely miscible with cannabinoids. In MCTs demonstrate an ability to create emulsions of sufficiently small particle size that are stable for at least a plurality of months at a time. For these reasons MCTs can be used as embodiments of carrier oils for development of the cannabinoid emulsion system. Illustrative examples of MCT oils can include, but are not limited to: caproic acid (C6), caprylic acid (C8), capric acid (C10), lauric acid (C12), fractionated coconut oil, Captex 300, Miglyol 810, Miglyol 812, Neobee M-5, or combinations thereof.

Examples of ratios of cannabis oil:MCT carrier oil can include, but are not limited to: 1:0 to 1:10000, or 1:0 to 1:50, or 1:1 to 1:10. Specific examples include, but are not limited to: 1:1 (65-95% THC or CBD cannabis oil:MCT fractionated coconut oil); 1:2 (65-95% THC or CBD cannabis oil:MCT fractionated coconut oil); 1:5 (65-95% THC or CBD cannabis oil:MCT fractionated coconut oil); or 1:10 (65-95% THC or CBD cannabis oil:MCT fractionated coconut oil). Such ratios can create emulsified cannabinoids that could be turned into self-emulsifying powders as described herein.

According to other embodiments, the present powdered component can demonstrate high bioavailability using LCTs. In an effort to increase bioavailability, long chain triglycerides (LCTs) can be utilized as a primary carrier oil. LCTs exhibit substantial, and optionally complete miscibility with cannabinoids. In addition they can create emulsions of sufficiently small particle sizes that are stable (i.e., remain in an emulsion without substantially settling or agglomerating to form a population of larger particle sizes) for at least a plurality of months at a time. Additionally, LCTs have slower digestion by enzymes than MCTs, resulting in protection of actives from degradation and enhanced bioavailability. For these reasons LCTs can be utilized as carrier oils for development of the cannabinoid emulsion systems. Examples of LCT oils can include, but are not limited to: myristic acid (C14), palmitic acid (C16), palmitoleic acid (C16, 1 double bond), stearic acid (C18), oleic acid (C18, 1 double bond), linoleic acid (C18, 2 double bonds), linolenic acid (C18, 3 double bonds), eicosapentaenoic acid (EPA) and docosahexaenoic acid (DHA). Further, alternate embodiments of LCTs can include, but are not limited to vegetable oil: corn oil, soybean oil, safflower oil, coconut oil, olive oil, cottonseed oil, palm oil, peanut oil, sesame oil, sunflower oil, rapeseed oil, canola oil, avocado oil, hemp oil, almond oil, brazil nut oil, cashew oil, hazelnut oil, macadamia nut oil, pecan oil, pine nut oil, pistachio oil, walnut oil, flaxseed oil, cocoa butter, grapeseed oil, rice bran oil, and any other relevant vegetable oils, optionally in combination with at least one other relevant oil.

Specific embodiments of LCT ratios include, but are not limited to ratios of cannabis oil:LCT carrier oil of: 1:0 to 1:10000, or 1:0 to 1:50, or 1:1 to 1:10. More specific examples include, but are not limited to:

1:1 (65-95% THC or CBD cannabis oil:olive oil),

1:2 (65-95% THC or CBD cannabis oil:olive oil),

1:5 (65-95% THC or CBD cannabis oil:olive oil),

1:10 (65-95% THC or CBD cannabis oil:olive oil),

1:1 (65-95% THC or CBD cannabis oil:sesame oil),

1:2 (65-95% THC or CBD cannabis oil:sesame oil),

1:5 (65-95% THC or CBD cannabis oil:sesame oil),

1:10 (65-95% THC or CBD cannabis oil:sesame oil),

1:1 (65-95% THC or CBD cannabis oil:vegetable oil),

1:2 (65-95% THC or CBD cannabis oil:vegetable oil),

1:5 (65-95% THC or CBD cannabis oil:vegetable oil), and

1:10 (65-95% THC or CBD cannabis oil:vegetable oil).

Such ratios can be utilized to create emulsified cannabinoids that could be turned into self emulsifying powders as described herein.

Another embodiment of a delivery system that has a rapid onset of action in addition to high bioavailability includes a combination of at least one LCT and at least one MCT. MCTs have the benefit of faster digestion and release of cannabinoids for absorption in the body. LCTs have the benefit of protecting cannabinoids from enzymatic degradation resulting in higher bioavailability in the body. By controlling the ratio of MCTs to LCTs a tradeoff of higher bioavailability and faster onset can be adjusted. Illustrative embodiments of the ratio of MCT to LCT for the oil carrier include, but are not limited to MCTs:LCTs=1:1, or 2:1, or 3:1, or 4:1, or 5:1, etc.; or 1:2, or 1:3, or 1:4, or 1:5, etc. An illustrative example of a range of ratios includes, but is not limited to 1:0 to 0:1.

Other carriers used in lipid based oral formulations that solubilize cannabinoids can be used according to alternate embodiments. Examples include, but are not limited to: propylene glycol esters (propylene glycol monocaprylate, propylene glycol monolaurate), fatty acids (cis-9 octadecanoic acid, hexadecanoic acid, octadecanoic acid, z,z-9-12-octadecadienoic acid), mono/diglycerides (glyceryl caprylate/caprate, glyceryl monocaprylate, glycerol monooleate), lipid mixtures (saturated C8-C18 triglycerides [Gelucire 33/01], MCTs+phosphatidylcholine [Phosal 53 MCT], phosphatidylcholine+safflower oil/ethanol [Phosal 75 5A]). Additionally, structured lipids that are triglycerides composed of both long and medium chain fatty acids esterified to the same glycerol backbone can be used as the carrier oil. These triglycerides have the advantage of faster hydrolysis, similar to MCTs, as well as the protection ability observed by LCTs.

Yet other embodiments of the present technology include the use of a cannabis oil emulsion without using a carrier oil. Such emulsions can also be encapsulated with a polymer coating and embedded into a matrix upon which they can be converted into a powder as described herein. However, emulsions made without carrier oil may not exhibit the same particle size and stability compared to when embodiments utilizing a carrier oil. Emulsions made using just cannabinoids have larger particle size and tend to observe phase separation over time. Additionally, emulsified cannabis powders free of carrier oil, made using the methodology described herein, tend to settle out of solution when reconstituted in aqueous liquids. Therefore, powders made from cannabis oil emulsions devoid of a carrier oil can be best suited for instant drinks and dry dosage forms, for example. Example of emulsions made without carrier oil include, but are not limited to:

1-15% cannabis oils load (60-90% THC; or 60-99% CBD; or 30-45% THC: 30-50% CBD),

1-15% emulsifier (gum arabic or modified gum arabic or modified starch, or whey protein isolate), and 70-98% water.

Such emulsions have been successfully combined with aqueous maltodextrin then dehydrated and milled into a free flowing powder. The resulting powder had the following formula 2 parts maltodextrin, 1 part cannabinoids and 1 part emulsifier. The resulting powder had concentrations of 1-200 mg/g THC or 1-200 mg/g CBD or 1-200 mg/g THC:CBD. The powder was then dissolved in aqueous solutions to create cannabinoid emulsions that were stable for a minimum of 2 days and have observed stability over 1 week.

Antioxidant/Chelator/Stabilizer Additive

Cannabinoids (Terpenes, Essential Oils) are susceptible to oxidation and hydrolysis. Dissolving the active ingredients in a carrier oil and encapsulating them in an emulsifier/surfactant can significantly improve the stability of the cannabinoids. However, overtime it is possible for cannabinoids to be exposed to oxygen, hydrogen ions (acids, water), in addition to any other environmental factors that will cause their degradation.

Organic bases can be used to prevent the degradation of the cannabinoids. These organic bases include, but are not limited to: butyl hydroxyl anisole (BHA), butyl hydroxyl toluene (BHT) and sodium ascorbate; at concentrations between 0.001 to 5% w/w in the emulsion system, for example. Organic bases such as the following can improve the stability of cannabinoids from chemical degradation for up to 2 years: BHA 0.001 to 5% w/w of emulsion system, BHT 0.001 to 5% w/w of emulsion system, and combinations of BHA and BHT can also be used.

Antioxidants can be used to prevent or at least inhibit or mitigate the degradation of cannabinoids from oxidation. Antioxidants include: ethanol, polyethylene glycol 300, polyethylene glycol 400, propylene glycol, propylene carbonate, N-methyl-2-pyrrolidones, dimethylacetamide, dimethyl sulfoxide, hydroxypropyl-P-cyclodextrins, sulfobutylether-β-cyclodextrin, a-cyclodextrin, HSPC phospholipid, DSPG phospholipid, DMPC phospholipid, DMPG phospholipid, ascorbyl palmitate, butylated hydroxy anisole, butylatedhydroxy anisole, propyl gallate, a-tocopherol, γ-tocopherol, propyl gallate, lecithin, Vitamin E tocopherol, sesamin, sesamol, sesamolin, alpha tocopherol, ascorbic acid, ascorbyl palmitate, fumaric acid, malic acid, sodium metabisulfite and EDTA. Specific antioxidant examples include, but are not limited to:

Ascorbic Acid: 0.001 to 5% w/w of emulsion system, Vitamin E Tocopherol: 0.001 to 5% w/w of emulsion system, Tocopherol: 0.001 to 5% w/w of emulsion system, and combinations of ascorbic acid, vitamin E tocopherol and tocopherol can be used for this invention.

Chelating agents can prevent or at least mitigate the degradation of cannabinoids from metal ions in solution. Chelating agents include, but are not limited to: Ethylenediaminetetraacetic acid (EDTA), phosphoric acid, polyphosphates, polysaccharides, citric acid and any combination thereof.

Many of the emulsions herein can be made using emulsifiers with a high microbial load. Additionally, oil-in-water emulsions are aqueous in nature and susceptible to microbial growth. Preservatives can be used to prevent microbial spoilage. These preservatives include: methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, propylene glycol, benzaldehyde, butylated hydroxytoluene, butylated hydroxyanisole, formaldehyde donors, essential oils, citric acid, monoglyceride, phenol, mercury components and any combination thereof. Specific examples include, but are not limited to:

Sodium Benzoate and Potassium Sorbate: 0.001-1% prevent microbial growth (4 months at least). Additionally, the pH of the emulsion can be lowered to prevent or retard microbial growth. Lowering the pH below 4.0 is sufficiently low enough to prevent microbial growth for a minimum of 1 month.

Preservatives and/or stabilizers can be added prior to emulsion formation or after emulsion formation. Depending on the nature of the preservative/stabilizer it will be contained in either the oil phase, interfacial layer, or the aqueous continuous phase. The emulsion containing preservative and/or stabilizer can be combined in aqueous solution of polymer. This polymer network can have the aqueous fraction removed by dehydration, evaporation, filtering, etc. The resulting dry polymer network has preservative and stabilizer embedded in it, which can then be powdered or milled. The resulting powder is dissolvable and self-emulsifying when reintroduced into an aqueous environment. Once dissolved the preservatives and stabilizers are released into solution imparting their properties into the aqueous system. This allows beverage manufacturers the ability to instantly create shelf stable cannabis infused beverages. Beverages made this way can resist microbial growth and chemical degradation for a minimum of 3 months.

Particle Density Modifier Additive

Emulsion systems observe accelerated phase separation when there is a difference in density between the oil and aqueous phases. If the emulsified oil phase is less dense than the continuous phase it will rise to the top resulting in creaming. Alternatively if the emulsified oil is more dense than the continuous phase it will settle out resulting in sedimentation. It is possible to match the density of the oil phase to that of the continuous phase using the appropriate weighting agents to retard instability due to phase separation.

The density of the oil phase in the emulsion can be increased by introducing a weighting agent. Appropriate weighting agents include but are not limited to ester gum, glycerol ester of wood rosin, dammar gum and sucrose acetoisobutyrate. The density of the oil phase can be reduced by diluting the cannabis oil/carrier oil with an oil of lower density such as essential oils (orange oil=0.844 g/ml), aromatics or alcohols (ethanol, sugar alcohols). Illustrative embodiments of the oil phase density include, but are not limited to: 1 g/ml=water; 1.1-1.4 g/ml=carbohydrate rich aqueous solutions, glycerol; 1.1-1.5 g/ml=syrups, honey; 0.7-0.95 g/ml=alcohols, oil systems; and >1.5 g/ml=carbohydrates and sugars. Weighting agents can be introduced into the oil phase to match these densities.

When the emulsified cannabinoid powder described herein is reconstituted in an aqueous environment both creaming and sedimentation are possible over time. Generally speaking when cannabis oils, with purities less than 75% cannabinoids, are emulsified without a carrier the resulting powder tends to sediment. The following embodiments reduce the rate of sedimentation when the emulsified cannabis powder is dissolved in aqueous solution:

Creating the emulsion with the following cannabis oil to carrier oil ratios;

1:1 to 1:9 (Cannabis oil:Carrier oil); and

Carrier oils: MCTs, LCTs, and combinations thereof.

After dissolution: emulsions stable for a minimum of 7 days and have observed stability for over 2 weeks. It has further been observed that when the ratio of carrier oil to cannabis oil is sufficiently high that creaming occurs when the powder is dissolved in an aqueous liquid. This typically occurs at cannabis oil to carrier oil ratios above 1:5. This can be fixed by adding gum ester to the oil phase to achieve a density of 1 g/ml prior to emulsification.

Emulsifier Additive

As discussed above, oil-in-water emulsions are capable of drastically increasing the bioavailability of lipophilic drugs compared to aqueous suspensions of the drugs or when the drugs are administered in carrier oils neat. Additionally, emulsions made using emulsifiers and/or surfactants offer a method of encapsulation for active drug components that protects them from environmental stresses that may cause unwanted degradation. Depending on the ingredients and processing conditions, emulsions can be created with specific particle sizes ranging from several microns down to nanometers with both high and low polydispersity. In this way cannabis based emulsions can be tailor made for specific applications. For instance to reduce onset times fine emulsion particles can be made with high surface areas that facilitate rapid uptake in the body. Additionally, certain emulsifiers such as modified gum arabic and modified starch can protect active ingredients from oxidation and hydrolysis.

Emulsifiers and surfactants are responsible for encapsulating the oily fraction and partitioning it from the aqueous phase. Depending on the type of emulsifier/surfactant used emulsions can be made from micelles or liposomes. Micelles have the oily fraction contained in the core, encapsulated in emulsifier/surfactant that partitions the oil phase from the aqueous phase. Liposomes are bilayer systems where an aqueous core is surrounded by a bilayer system containing the oil phase inside the bilayer.

The type of emulsifier/surfactant chosen has an impact on the particle size, emulsion stability, drug release profile and flavor profile. Additionally, the amount of emulsifier used has an impact on the particle size, the release profile and the encapsulation efficiency. Semi-natural and synthetic emulsifiers/surfactants can be used to create the emulsion systems described herein. Such emulsions can have sufficiently-small particle sizes that impart intrinsic emulsion stability and high dispersibility with high surface area that facilitates rapid uptake in the body.

### Emulsifier Embodiments

Modified starch (Purity Gum) is an example of an emulsifier for creating cannabinoid emulsions. Modified starch is soluble in hot and cold aqueous liquids. During emulsion preparation it is dissolved in the aqueous fraction and the aqueous fraction is combined with the oil fraction prior to emulsification. Emulsions made using modified starch have particle sizes between 50 nm to 20 microns, for example. Oil loads of 1-30% w/w are possible using modified starch at emulsifier:oil ratios of 1:1-1:4. Modified starch in the appropriate concentrations promotes complete encapsulation of the oil particles with a high encapsulation efficiency. A high encapsulation efficiency is defined by over 70% of the cannabinoids being contained in the emulsion phase, although alternate embodiments achieve encapsulation efficiency of at least 90%. Emulsions made using purity gum demonstrate stability for at least 3 months with no apparent phase separation and over 80% of the original cannabinoid concentration being maintained. Further after 3 months the particle size remains less than 20 microns, and less than 800 nm. The emulsions prepared using modified starch are stable under the following conditions: pH 1-10, under heating (100° C. for 5-10 min), under freezing and thawing conditions (0° C. and 25° C.), ultrasonic vibration during homogenization, and under dehydration/lyophilization conditions. Emulsions made using modified starch have also demonstrated protection of cannabinoids from hydrolysis and oxidation for over 3 months. Additionally, bitter and harsh flavor tones of cannabis oils are drastically subdued when modified starch is used as the emulsifier. In this regard, purity gum acts as a bitter suppressant in the cannabinoid emulsions. A specific embodiment modified starch (purity gum) is:

Ratio of cannabis oil to carrier oil=1:0, 1:1, 1:2, 1:5, 1:10, etc.

Type of cannabis oil: 60-90% THC cannabis oil, 60-99% CBD oil, Combinations of THC:CBD (1:1, etc.),

Carrier oils: MCT or olive oil or no carrier used,

Oil load: 0.5-30% w/w, or optionally 1-15% w/w,

Emulsifier: 0.25-50% w/w, or optionally 1-15% w/w Purity Gum,

Water phase: 20-99% w/w, water makes up the difference of the emulsion,

Particle size: 50 nm-20 microns, or optionally 100-800 nm,

Cannabinoid concentration: 0.1-300 mg/g, or optionally 5-150 mg/g,

Encapsulation efficiency: at least 70%, or optionally at least 80%, or optionally at least 99%

Emulsion stability (25 C): 1 week minimum, or at least 1 month, or at least 6-12 months.

Cannabinoid stability (25 C): 1 month maintains 80-90% original cannabinoids, but this stability can be maintained for 6-12 months.

Gum arabic is an emulsifier capable of creating stable cannabinoid emulsions. The emulsions can be made using gum arabic (TIC Pretested Gum Arabic Spray Dry Powder, TIC Pretested Gum Arabic BEV-101 GR Powder) and modified/quick hydrating gum arabic (TICAMULSION A-2010 Powder, TICAMULSION 3020, GUMPLETE GUM ARABIC S SD). The emulsification power of gum arabic largely depends on the arabinogalactan fraction, the arabinogalactoprotein fraction and the glycoprotein fraction. The emulsification ability of gum arabic is attributed to the arabinogalactoprotein fraction. Depending on the source of the gum arabic and if any modifications have made been made, gum arabic can vary widely in its emulsification ability.

According to another embodiment, organic (or regular) gum arabic is used as the emulsifier to make cannabinoid emulsions. Gum arabic is soluble in both hot and cold aqueous liquids. The gum arabic is dissolved in the aqueous phase and allowed to hydrate. Gum arabic is prepared in the aqueous fraction and then combined with the oil fraction during emulsification. Emulsions made using gum arabic have particle sizes between 50 nm to 20 microns, but the particle size using gum arabic can be between 100-800 nm after emulsification. Oil loads of 1-25% w/w are possible in this invention using gum arabic at emulsifier:oil ratios of 2:1-1:4. Gum arabic in the appropriate concentrations promotes complete encapsulation of the oil particles with high encapsulation efficiency. A high encapsulation efficiency is defined by over 70% of the cannabinoids being contained in the emulsion phase, but alternate embodiments have an encapsulation efficiency that is above 90%. The emulsions made using gum arabic may demonstrate stability for over 3 months with no apparent phase separation and over 80% of the original cannabinoid concentration being maintained. Further after 3 months the particle size is below 20 microns, or less than 800 nm for some embodiments. The emulsions prepared using gum arabic were stable under the following conditions: pH 1-10, under heating (100 C for 5-10 min), under freezing and thawing conditions (0 C and 25 C), ultrasonic vibration during homogenization, and under dehydration/lyophilization conditions. Emulsions made using gum arabic may also demonstrate protection of cannabinoids from hydrolysis and oxidation for over 3 months. Additionally, bitter and harsh flavor tones of cannabis oils are drastically subdued when gum arabic is used as the emulsifier. In this regard, gum arabic acts as a bitter suppressant in the cannabinoid emulsions. Other specific examples of the gum Arabic include, but are not limited to:

Ratio of cannabis oil to carrier oil=1:0, 1:1, 1:2, 1:5, 1:10, etc.,

Type of cannabis oil: 60-90% THC cannabis oil, 60-99% CBD oil, Combinations of THC:CBD (1:1, etc.),

Carrier oils: MCT or olive oil or no carrier used,

Oil load: 0.5-25% w/w, or 1-15% w/w,

Emulsifier: 0.25-50% w/w, or 1-15% w/w gum Arabic,

Water phase: 25-99% w/w, water makes up the difference of the emulsion,

Particle size: 50 nm-20 microns, or 100-800 nm

Cannabinoid concentration: 0.1-250 mg/g, or 5-150 mg/g

Encapsulation efficiency: at least 70%, or at least 80%, or at least 99%

Emulsion stability (25 C): 1 week minimum, but extended stability periods of at least 1 month, or 6-12 months were also observed.

Cannabinoid stability (25 C): 1 month maintains 80-90% original cannabinoids, but 6-12 months was also observed.

Gum arabic modified with octenyl succinic acid (modified gum arabic: TICAMULSION 3020, GUMPLETE GUM ARABIC S SD) can be used according to alternate embodiments as the emulsifier to make cannabinoid emulsions. Modified gum arabic is capable of emulsifying higher oil loads and achieving smaller particle sizes compared to unmodified gum arabic. It is also protects cannabinoids from oxidation better than both gum arabic and modified starch. Modified gum arabic is soluble in both hot and cold aqueous liquids. Modified gum arabic is prepared in the aqueous fraction and then combined with the oil fraction during emulsification. Emulsions made using modified gum arabic have particle sizes between 50 nm to 20 microns. Oil loads of 1-30% w/w are possible using modified gum arabic at emulsifier:oil ratios of 2:1-1:4. Modified gum arabic in the appropriate concentrations promotes complete encapsulation of the oil particles with high encapsulation efficiency. A high encapsulation efficiency is defined by over 70% of the cannabinoids being contained in the emulsion phase, but encapsulation efficiencies above 90% are also achieved. The emulsions made using modified gum arabic have demonstrated stability for over 3 months with no apparent phase separation and over 80% of the original cannabinoid concentration being maintained. Further after 3 months the particle size is less than 20 microns, and optionally less than 800 nm. The emulsions prepared using modified gum arabic are stable under the following conditions: pH 1-10, under heating (100° C. for 5-10 min), under freezing and thawing conditions (0° C. and 25° C.), ultrasonic vibration during homogenization, and under dehydration/lyophilization conditions. Emulsions made using modified gum arabic promote protection of cannabinoids from hydrolysis and oxidation for over 3 months. Additionally, bitter and harsh flavor tones of cannabis oils are drastically subdued when modified gum arabic is used as the emulsifier. In this regard, modified gum arabic acts as a bitter suppressant in the cannabinoid emulsions. Illustrative examples of the modified gum Arabic include, but are not limited to:

Ratio of cannabis oil to carrier oil=1:0, 1:1, 1:2, 1:5, 1:10, etc.,

Type of cannabis oil: 60-90% THC cannabis oil, 60-99% CBD oil, Combinations of THC:CBD (1:1, etc.),

Carrier oils: MCT or olive oil or no carrier oil used,

Oil load: 0.5-30% w/w, or 1-15% w/w,

Emulsifier: 0.25-50% w/w, or 1-15% w/w modified gum Arabic,

Water phase: 20-99% w/w, water makes up the difference of the emulsion,

Particle size: 50 nm-20 microns, or 100-800 nm,

Cannabinoid concentration: 0.1-300 mg/g, or 5-150 mg/g,

Encapsulation efficiency: at least 70%, or at least 80%, or at least 99%

Emulsion stability (25 C): 1 week minimum, or at least 1 month, or 6-12 months.

Cannabinoid stability (25 C): 1 month maintains 80-90% original cannabinoids, or at least 6-12 months.

Whey protein isolate (WPI) is another example of an emulsifier that can be used to create the encapsulated cannabinoids of this invention. WPI is soluble in hot and cold aqueous liquids. During emulsion preparation it is dissolved in the aqueous fraction and the aqueous fraction is combined with the oil fraction prior to emulsification. Emulsions made using WPI have particle sizes between 50 nm to 20 microns. WPI is capable of emulsifying oils at low usage levels. An oil load of 1 to 20% w/w can be emulsified with 0.25 to 10% w/w WPI. The type of WPI is important for the emulsion system and intended applications. Many emulsion systems are subjected to low pH and high temperature when used in beverage formulas. Therefore it is advantageous to use a WPI emulsifier that is stable to both acidic conditions and high temperatures (Hilmar 4020 WPI). A wide variety of WPI can be used to create emulsions and is not limited to heat and pH stable WPI. WPI also has the advantage of being directly lyophilized. Therefore, cannabinoids that have been encapsulated using WPI can either be directly lyophilized or embedded in a polymer network and lyophilized to create a self emulsifying cannabis powder. Cannabinoids encapsulated with WPI demonstrate significant taste masking. In many regards, cannabis oil that has been encapsulated with WPI is virtually flavorless. Additionally, the introduction of WPI in the emulsion system has the potential to introduce a significant source of nutritional protein for applications in sports drinks and supplements. Illustrative examples of compositions including WPI include, but are not limited to:

Ratio of cannabis oil to carrier oil=1:0, 1:1, 1:2, 1:5, 1:10, etc.,

Type of cannabis oil: 60-90% THC cannabis oil, 60-99% CBD oil, Combinations of THC:CBD (1:1, etc.),

Carrier oils: MCT or olive oil or no carrier used,

Oil load: 0.5-20% w/w, or 1-15% w/w,

Emulsifier: 0.25-30% w/w, or 1-10% w/w WPI,

Water phase: 50-99% w/w, water makes up the difference of the emulsion,

Particle size: 50 nm-20 microns, or 100-800 nm

Cannabinoid concentration: 0.1-200 mg/g, or 5-150 mg/g

Encapsulation efficiency: at least 70%, or at least 80%, or at least 99%

Emulsion stability (25 C): 1 week minimum, or at least 1 month, or at least 6-12 months,

Cannabinoid stability (25 C): 1 month maintains 80-90% original cannabinoids, or at least at 6-12 months.

The emulsions containing cannabis can then be directly lyophilized. The resulting solid can be milled into a free flowing powder. The powder can dissolved in aqueous liquids creating an emulsion of WPI encapsulated cannabinoids.

Polymer based emulsifiers can be utilized to create encapsulated cannabinoid emulsions. However, it is possible to create cannabinoid emulsions using a wide variety and combination of different emulsifiers and surfactants. These include but are not limited to: Q-naturale (quillaja saponins), polysorbates (Tween 20, 60, 80), Span (20, 60, 80, 83, 85, 120), vegetable lecithins (phospholipids), polyethylene glycols (PEG 300, 400, 600) polyethylene glycol esters, polyethylene glycol ethers, polypropylene glycol ethers, polyol esters, poloxamers.

Emulsion Formulation

The method of emulsification and the order of addition can impact properties such as stability, particle size and encapsulation efficiency. Embodiments of the present method can optionally use a top-down approach to create cannabinoid based emulsions. The cannabinoid rich oil fraction is dispersed into smaller droplets on the order of nanometers to micrometers using a high energy input. The emulsifiers of this invention self assemble at the interface of the oil droplets creating an interfacial barrier that encapsulates the oil and active ingredients.

The formulation, type of emulsifier and energy input, control the particle size and properties of the emulsion. The following formulations have demonstrated emulsions with particle sizes between 50 nm and 20 microns, or optionally between 100 and 800 nm. The emulsions are stable for over 3 months; maintaining particle sizes between 50 nm and 20 microns with no, or limited phase separation.

The following procedure was used to create the emulsions of this invention unless otherwise stated:

Oil phase prepared,

Cannabis oils are heated (60-90° C.),

Various cannabis oils and essential oils are mixed to achieve desired cannabinoid and terpene ratios,

Carrier oil, if used is heated (60-90° C.),

The cannabis oils are mixed into the carrier oil,

Additional stabilizers or additives are added if necessary,

Aqueous phase prepared,

The appropriate emulsifier or combination of emulsifiers are hydrated in water (overnight at 4-25° C.),

Additional stabilizers or additives are added if necessary,

The oil phase and aqueous phase are mixed together (25-90° C.),

Shear mixing is used to create a coarse emulsion,

The coarse emulsion is homogenized using an ultrasonic homogenizer (either batch or continuous),

Sonication amplitude: 50-100 upp,

Sonication residence time: 1-30 min,

Sonication temperature: 0-100° C.,

Following sonication any additional additives or preservatives are added as necessary,

Below are examples of emulsions prepared using this procedure:

### Example 1

5-10% w/w oil load: 1 part 70-90% THC cannabis oil, 1 part carrier oil (MCT and/or olive oil),

2.5-10% w/w Emulsifier: Purity Gum or gum arabic or modified gum arabic (TICAMULSION 3020),

80-92.5% w/w water,

Sonicated 90-100 upp, 5 min at 40° C.,

THC concentration: 17.5 mg/g to 45 mg/g,

Encapsulation efficiency: 95%+,

Cannabinoid stability (25° C.): 5 months+,

Emulsion stability (25° C.): 5 months+,

### Example 2

5-10% w/w oil load: 70-90% THC cannabis oil,

2.5-10% w/w Emulsifier: Purity Gum or gum arabic or modified gum arabic (TICAMULSION 3020),

80-92.5% w/w water,

Sonicated 90-100 upp, 5 min at 40° C.,

THC concentration: 35 mg/g to 90 mg/g,

Encapsulation efficiency: 95%+,

Cannabinoid stability (25 C): 5 months+,

Emulsion stability (25 C): 3 months+,

### Example 3

5-10% w/w oil load: 1 part 70-99% CBD cannabis oil, 1 part carrier oil (MCT and/or olive oil),

2.5-10% w/w Emulsifier: Purity Gum or gum arabic or modified gum arabic (TICAMULSION 3020),

80-92.5% w/w water,

Sonicated 90-100 upp, 5 min at 40° C.,

CBD concentration: 17.5 mg/g to 49.5 mg/g,

Encapsulation efficiency: 95%+,

Cannabinoid stability (25 C): 5 months+,

Emulsion stability (25 C): 5 months+,

### Example 4

5-10% w/w oil load: 70-99% CBD cannabis oil,

2.5-10% w/w Emulsifier: Purity Gum or gum arabic or modified gum arabic (TICAMULSION 3020),

80-92.5% w/w water,

Sonicated 90-100 upp, 5 min at 40° C.,

CBD concentration: 35 mg/g to 100 mg/g,

Encapsulation efficiency: 95%+,

Cannabinoid stability (25 C): 5 months+,

Emulsion stability (25 C): 3 months+,

### Example 5

5-10% w/w oil load: 1 part cannabinoid oil of varying THC and CBD concentrations, 1 part carrier oil (MCT and/or olive oil),

2.5-10% w/w Emulsifier: Purity Gum or gum arabic or modified gum arabic (TICAMULSION 3020),

80-92.5% w/w water,

Sonicated 90-100 upp, 5 min at 40° C.,

THC concentration: 0.1 mg/g to 45 mg/g,

CBD concentration: 0.1 mg/g to 49.5 mg/g,

Encapsulation efficiency: 95%+,

Cannabinoid stability (25 C): 5 months+,

Emulsion stability (25 C): 5 months+,

### Example 6

5-10% w/w oil load: Cannabinoid oil of varying THC and CBD concentrations,

2.5-10% w/w Emulsifier: Purity Gum or gum arabic or modified gum arabic (TICAMULSION 3020),

80-92.5% w/w water,

Sonicated 90-100 upp, 5 min at 40° C.,

THC concentration 0.1 mg/g to 90 mg/g,

CBD concentration: 0.1 mg/g to 100 mg/g,

Encapsulation efficiency: 95%+,

Cannabinoid stability (25 C): 5 months+,

Emulsion stability (25 C): 3 months+,

### Example 7

5-10% w/w oil load: 1 part cannabinoid oil of varying THC and CBD concentrations, 1 part carrier oil (MCT and/or olive oil),

1.5-3% w/w Emulsifier: whey protein isolate (Hilmar 4020),

87-93.5% w/w water,

Sonicated 90-100 upp, 5 min at 40° C.,

THC concentration: 0.1 mg/g to 45 mg/g,

CBD concentration: 0.1 mg/g to 49.5 mg/g,

Encapsulation efficiency: 95%+,

Cannabinoid stability (25 C): 5 months+,

Emulsion stability (25 C): 5 months+,

### Example 8

5-10% w/w oil load: Cannabinoid oil of varying THC and CBD concentrations,

1.5-3% w/w Emulsifier: whey protein isolate (Hilmar 4020),

87-93.5% w/w water,

Sonicated 90-100 upp, 5 min at 40° C.,

THC concentration 0.1 mg/g to 90 mg/g,

CBD concentration: 0.1 mg/g to 100 mg/g,

Encapsulation efficiency: 95%+,

Cannabinoid stability (25 C): 5 months+,

Emulsion stability (25 C): 3 months+,

All of the above examples resulted in stable emulsions. They remained stable and easily dispersed when diluted in aqueous solutions of acidic pH, basic pH, high ionic strength and carbohydrate and/or polymer rich solutions, or combinations thereof. They also were stable through freezing/thawing conditions and at elevated temperatures (100° C.).

The following high energy methods can be used to create the cannabinoid emulsions of this invention:

High pressure homogenization/Microfluidic homogenization—a homogenizer is used to force a stream of a primarily liquid sample through a system which subjects the stream and sample to forces intended to homogenize the sample and/or reduce the particle sizes of any components within the sample.

Ultrasonic homogenization—a transducer containing piezoelectric crystals that convert electric energy into high-frequency mechanical motion that is amplified and emitted by a probe into the sample.

High shear mixing—a rotor or impeller, together with a stationary component known as a stator, or an array of rotors and stators, is used either in a tank containing the solution to be mixed, or in a pipe through which the solution passes, to create shear on the medium to be homogenized.

Colloidal milling—similar to high shear mixing, a rotor is turned at high speeds (e.g., 2000-18000 RPM) adjacent to a stator to reduce the particle size of components suspended in a liquid.

Of the above high-energy methods, high pressure homogenization and ultrasonic homogenization were found to create smaller particle sizes than the other methods and can easily be scaled up for commercial production.

Polymer Matrices

The cannabinoids can be encapsulated into a biodegradable polymer matrix or polymer assemblage. The polymer matrix can serve many functions including: improving the stability of the cannabinoid emulsion, ease of handling, creating new dosage forms, controlling drug pharmacokinetics and controlling drug delivery.

Maltodextrin Entrapment—Maltodextrin is an illustrative example of a suitable polymer matrix. In one embodiment, the encapsulated cannabinoid emulsion can be combined with an aqueous solution of maltodextrin. The emulsion and the aqueous maltodextrin are completely, or at least substantially miscible. In this way the encapsulated cannabinoids can be homogeneously distributed or dispersed throughout the solution. The water fraction of the mixture can then be removed through dehydration of other technique to create a solid dehydrated mass. Once dehydrated, the maltodextrin polymers and the encapsulated cannabinoids are cast into place with an even distribution of cannabinoids throughout. Casting of encapsulated cannabinoids into maltodextrin can be performed in a manner that prevents aggregation of particles and maintains adequate particle sizes of the encapsulated cannabinoids. Encapsulated cannabinoid particle sizes can be established and maintained between 50 nm and 20 microns.

The dehydrated maltodextrin agglomeration with encapsulated cannabinoids can be shelf stable from degradation and microbial growth. Cannabinoids quickly oxidize with exposure to oxygen and tend to hydrolyze in the presence of moisture. The method of encapsulating the cannabinoids followed by embedding them in a dry polymer network, which shields the encapsulated cannabinoid from air and humidity, aids in protecting the cannabinoids from degradation. Additionally, removal of water aids in preventing microbial growth. Cannabinoids can prepared this way to maintain their integrity (potency, consistency, etc.) for over 1 month, but can be prepared to maintain their integrity for over 6 months, or for over 2 years.

A number of methods can be used to dehydrate or remove water from the emulsion/maltodextrin solution. These include but are not limited to: evaporation via microwave, convection oven, vacuum oven etc., lyophilization, spray drying, shell freezing. However, certain drying methods are believed to prevent particle aggregation/coalescence, maintain particle sizes, and maintain a high encapsulation efficiency. Such methods of water removal are lyophilization or spray drying. Lyophilization and/or spray drying the cannabinoid emulsions/maltodextrin solution results in a dry solid mass that can be milled into a free flowing powder.

Lyophilization, for example, is capable of dehydrating the system at temperatures below 100° C. using sub-atmospheric pressures. This preserves the entrapment and encapsulation of volatile compounds such as terpenes that begin to boil at about 120° C. at 1 atm. Lyophilization can also control the dissolution properties of the resulting powder. The pore size, porosity, and polymer composition factor into how quickly the powder will dissolve in both hot and cold aqueous liquids. The polymer composition is described above and the pore size and porosity are controlled by the initial water content and the lyophilization conditions. The initial water content of the system can be between 10% water by weight and 99% water by weight, but all sub-ranges therein are also within the scope of the present disclosure. Lyophilization can occur between 200 mTorr and atmospheric pressure and temperatures between −80° C. and 100° C., for example, until the system is completely or substantially dehydrated. The resulting pore sizes are between 100 nm and 1 mm, with overall porosities of 10% to 90% of interconnected void space. This results in a powder that is quickly hydrated and self emulsifies. The resulting powder with dissolve on time scales between 10 seconds and 5 minutes when exposed to the aqueous environment.

The dehydrated maltodextrin agglomeration embedded with encapsulated cannabinoids can then be milled into a free flowing powder having a bulk particle sizes of 200 nm to 3 mm, or particle sizes of 1 micron to 1000 microns, or particle sizes of 50 microns to 600 microns. Alternatively, the aqueous mixture of maltodextrin/emulsion can be spray dried directly into a free flowing powder resulting in similar powder particle sizes.

The resulting powder does not clump and rapidly dissolves in both hot and cold aqueous liquids. Rapid dissolving is defined by dissolution times of less than 10 minutes, or less than 5 minutes, or less than 3 minutes. This property is largely controlled by the drying procedure and degree of polymerization of the maltodextrin. The degree of polymerization of maltodextrin is reported as dextrose equivalents (DE), where DE=100/degree polymerization. Maltodextrin can be made to have DE values between 3 and 20. As the DE value decreases the polymer chain length of the maltodextrin increases. Further, as the DE decreases the following characteristics increase: molecular weight, viscosity, solubility, cohesiveness, film-forming properties, prevention of crystallization (cryoprotection). If the maltodextrin is completely soluble in aqueous liquids to allow for the dissociation of the polymer from the encapsulated cannabinoids. Further, freeze drying (or spray drying) the emulsion/maltodextrin solution maintains a bulk encapsulated cannabinoid particle sizes of about 50 microns to about 20 microns.

Creating a maltodextrin film around encapsulated particles can reduce the encapsulation efficiency and lower the overall yield for drug products. However, an encapsulation efficiency of at least 70%, or at least 90%, or at least 95%+can be achieved and maintained by establishing a the DE of about 5 to about 15, and freeze drying the encapsulated cannabinoids.

As mentioned above, the dehydrated maltodextrin agglomeration embedded with particles can be dissolved by aqueous solutions. The powdered maltodextrin/encapsulated complex is configured to disperse rapidly in aqueous solutions without clumping. The powdered complex dissolves rapidly with minimal agitation, between 10 seconds and 7 minutes in cold water (4-20° C.), between 1 and 7 minutes tepid water (20-50° C.), between 5 seconds and 3 minutes in hot water (50-100° C.), and between 30 seconds and 2 minutes in saliva (approximately 37° C.), etc. Once dissolved the maltodextrin polymers completely dissociate from the encapsulated cannabinoids liberating them from the agglomeration. The liberated encapsulated cannabinoids can have particle sizes between 50 nm and 20 microns in aqueous solution. After dissolution the encapsulated cannabinoids evenly disperse into the aqueous phase. The resulting emulsion is stable for a minimum of 24 hours, but the stability can remain for longer than 1-3 months, or in excess of 6-24 months from the formation of the emulsion.

As a specific example of a process for embedding encapsulated cannabinoids in maltodextrin matrix:

emulsion preparation (procedure above) is performed

Cannabinoid concentration: 0.1-400 mg/ml

Particle Size: 50-2000 nm

Water content: 50-95% w/w

Oil content: 1-20% w/w

Emulsifier content: 0.25-20% w/w

Aqueous maltodextrin preparation

Dissolve maltodextrin in water (25-50 C)

DE: 5-15

Maltodextrin Concentration: 20-80% w/w

Mixing maltodextrin solution into emulsion

Proportions of emulsion and maltodextrin (aq.) according to ratio

[Total Wt. MD: Total Wt. Emulsifier+Oil]

1:1-20:1

1:1-1:10

Dehydration/Evaporation

Freeze drying, spray drying, microwave, etc.

Milling/Grinding

Other examples of the powder compositions are tabulated in Table 1 below:

TABLE 1

| | % Cannabis | % Carrier | % | THC/CBD | Powder | Drying | |
|---|---|---|---|---|---|---|---|
| % MD | oil | oil | Emulsifier | concentration | particle size | Method | Notes |
| 50 | 25 | 0 | 25 | 80-200 mg/g | 500-2000 | Microwave | 70-90% THC retention |
| DE = 10 | 70-90% | | Mod. gum | THC | micron | | Self emulsify: stable 1 |
| | THC distil. | | arab. | | | | week then sediment |
| 50 | 25 | 0 | 25 | 90 mg/g THC | 500-2000 | Microwave | 70-90% THC retention |
| DE = 10 | 50% THC | | Mod. gum | | micron | | Self emulsify: stable 1 |
| | distil. | | arab. | | | | week then sediment |
| | | | | | | | Cannabis flavor |
| 50 | 25 | 0 | 25 | 80-300 mg/g | 500-2000 | Microwave | 70-90% CBD retention |
| DE = 10 | 70-99% | | Mod. gum | CBD | micron | | Self emulsify: stable 1 |
| | CBD distil. | | arab. | | | | week then sediment |
| 50 | 12.5 | 12.5 | 25 | 20-50 mg/g | 500-2000 | Microwave | 70-90% THC retention |
| DE = 10 | 70% THC | MCT | Mod. gum | THC | micron | | Self emulsify: stable 2 |
| | distil. | | arab. | | | | month (no separation) |
| | | | | | | | Flavorless |
| 50 | 12.5 | 12.5 | 25 | 50-70 mg/g | 500-2000 | Microwave | 70-90% CBD retention |
| DE = 10 | 90% CBD | MCT | Mod. gum | CBD | micron | | Self emulsify: stable 2 |
| | iso. | | arab. | | | | month (no separation) |
| | | | | | | | Flavorless |
| 50 | 12.5 | 12.5 | 25 | 20-50 mg/g | 500-2000 | Microwave | 70-90% THC retention |
| DE = 10 | 70% THC | Olive oil | Mod. gum | THC | micron | | Self emulsify: stable 2 |
| | distil. | | arab. | | | | month (no separation) |
| | | | | | | | Flavorless |
| 50 | 2.5 | 22.5 | 25 | 5-10 mg/g | 500-2000 | Microwave | 70-90% CBD retention |
| DE = 10 | 60% CBD oil | MCT | Mod. gum | CBD | micron | | Self emulsify: stable 2 |
| | | | arab. | | | | month (no separation) |
| | | | | | | | Flavorless |
| 66 | 8.25 | 8.25 | 16.5 | 10-40 mg/g | 500-2000 | Microwave | 70-90% THC retention |
| DE = 10 | 70% THC oil | MCT | Mod. gum | THC | micron | | Self emulsify: stable 2 |
| | | | arab. | | | | month (no separation) |
| | | | | | | | Flavorless |
| 75 | 6.25 | 6.25 | 12.5 | 10-30 mg/g | 500-2000 | Microwave | 70-90% THC retention |
| DE = 10 | 70% THC oil | MCT | Mod. gum | THC | micron | | Self emulsify: stable 2 |
| | | | arab. | | | | month (no separation) |
| | | | | | | | Flavorless |
| 50 | 18.75 | 18.75 | 12.5 | 30-70 mg/g | 500-2000 | Microwave | 70-90% THC retention |
| DE = 10 | 70% THC | Olive oil | Mod. gum | THC | micron | | Self emulsify: stable 2 |
| | distil. | | arab. | | | | month (no separation) |
| | | | | | | | Flavorless |

Polymer Matrices Other Than Maltodextrin

The present disclosure is not limited to maltodextrin, but can use a variety of polymer matrices to embed the encapsulated emulsion. Any polymers that are biodegradable and capable of maintaining the stability and particle sizes of the encapsulated cannabinoids can be utilized. The final form of the cannabinoid embedded polymer matrix can be dry, hydrated, agglomerated or cross-linked. The specific polymer chosen will control the drug delivery properties and applications of the encapsulated cannabinoids. The following polymers can be used as polymer matrices to embed the encapsulated cannabinoids: gelatin, pullulan, sodium alginate (both free and cross-linked), pectin, protein, polylactic acid, poly(lactic-co-glycolic) acid, poly(caprolactone), chitosan, cellulose (microcrystalline), polyglycolide, polylactide, polyhydroxobutyrate, hyaluronic acid, and collagen. Below are some procedures and examples of embedding the encapsulated cannabinoids in polymers other than maltodextrin.

Procedure for embedding encapsulated cannabinoids in gelatin matrix:

Emulsion preparation (procedure above)

Cannabinoid concentration: 0.1-400 mg/ml

Particle Size: 50-2000 nm

Water content: 50-95% w/w

Oil content: 1-20% w/w

Emulsifier content: 0.25-20% w/w

Aqueous polymer preparation

Dissolve gelatin in hot water (40-100 C)

Gelatin concentration: 0.25-20% w/w

Mixing gelatin solution into emulsion (40-100 C)

Proportions of emulsion and gelatin (aq) according to ratio

[Total Wt. Gelatin: Total Wt. Emulsifier+Oil]

1:1-20:1

1:1-1:10

Allow to cool and gel

Dehydration/Evaporation

Freeze drying, spray drying, microwave, etc.

Milling/Grinding.

Example powder formulas of encapsulated cannabinoids embedded in gelatin:

2-50% w/w gelatin

1-40% w/w cannabis oils (various concentrations of THC/CBD etc can be used)

0-40% w/w carrier oil (MCT, olive oil, etc.)

1-40% w/w emulsifier: purity gum or gum arabic or modified gum arabic or whey protein

Cannabinoid concentrations: 0.1 to 300 mg/g

Powder particle size: 500 to 2000 microns

The powder dissolved upon introduction into aqueous environments with temperatures above 30° C.

Procedure for embedding encapsulated cannabinoids in sodium alginate matrix:

emulsion preparation (procedure above)

Cannabinoid concentration: 0.1-400 mg/ml

Particle Size: 50-2000 nm

Water content: 50-95% w/w

Oil content: 1-20% w/w

Emulsifier content: 0.25-20% w/w

Aqueous polymer preparation

Dissolve sodium alginate in water (25-100 C)

Sodium alginate Concentration: 0.25-20% w/w

Mixing sodium alginate solution into emulsion (25-100 C)

Proportions of emulsion and sodium alginate (aq) according to ratio

[Total Wt. Sodium Alginate: Total Wt. Emulsifier+Oil]:

1:1-20:1

1:1-1:10

Cross linking of sodium alginate

Addition of aqueous calcium chloride

titrating 50 mmol to 1 molar calcium chloride until gelling of the sodium alginate matrix occurs.

Dehydration/Evaporation

Freeze drying, spray drying, microwave, etc.

Milling/Grinding

Example powder formulas of encapsulated cannabinoids embedded in sodium alginate:

2-50% w/w sodium alginate

1-40% w/w cannabis oils (various concentrations of THC/CBD etc can be used)

0-40% w/w carrier oil (MCT, olive oil, etc.)

1-40% w/w emulsifier: purity gum or gum arabic or modified gum arabic or whey protein

Cannabinoid concentrations: 0.1 to 300 mg/g

Powder particle size: 500 to 2000 microns

The powder dissolves upon introduction into acidic aqueous environments.

Procedure for embedding encapsulated cannabinoids in pectin matrix:

Emulsion preparation (procedure above)

Cannabinoid concentration: 0.1-400 mg/ml

Particle Size: 50-2000 nm

Water content: 50-95% w/w

Oil content: 1-20% w/w

Emulsifier content: 0.25-20% w/w

Aqueous polymer preparation

Dissolve pectin in water (25-100 C)

Pectin concentration: 0.25-50% w/w

Mixing pectin solution into emulsion (40-100 C)

Proportions of emulsion and pectin (aq) according to ratio

[Total Wt. Pectin: Total Wt. Emulsifier+Oil]

1:1-20:1

1:1-1:10

Gelling

Change in pH or addition of sugar to induce gelling of pectin matrix

Lower pH 1-5

or increase sugar concentration above 25 mmol

Dehydration/Evaporation

Freeze drying, spray drying, microwave, etc.

Milling/Grinding

Example powder formulas of encapsulated cannabinoids embedded in pectin:

2-50% w/w pectin

1-40% w/w cannabis oils (various concentrations of THC/CBD etc can be used)

0-40% w/w carrier oil (MCT, olive oil, etc.)

1-40% w/w emulsifier: purity gum or gum arabic or modified gum arabic or whey protein

Cannabinoid concentrations: 0.1 to 300 mg/g

Powder particle size: 500 to 2000 microns

Particle Size

The particle size of the emulsion affects the stability of the emulsion in addition to the pharmacokinetics of the cannabinoids. Sub-micron particles can be taken up by the majority of cells. There is a strong correlation between particle size and bioavailability in the body. For instance, some 100 nm drug particles are 2.5× more bioavailable than similar 1 micron particles and 6× more bioavailable than 10 micron particles. Additionally, particles have demonstrated to penetrate throughout the sub-mucosal layers in the mouth and intestine. In many ways the drug particle distribution can be varied by changing the particle size. Particle size can also be used to control the drug release profile, drug particles with smaller cores have to travel shorter distances to cross the interfacial layer and therefore are released quicker. Additionally, decreasing the particle size results in a larger collective surface area, which increases the dispersion of the drug increasing the rate of absorption.

Emulsions with particle sizes 50 nm to 20 microns can be created, but other embodiments have particle sizes from about 100 nm to about 10000 nm, or particle sizes from about 100 nm to about 1000 nm. The particles are embedded into a polymer matrix or agglomeration, both dry and hydrated, while maintaining particle sizes for encapsulated cannabinoids with minimal aggregation. For example, cannabinoid particle sizes from about 50 nm to about 20 microns, or about 100 nm to about 10000 nm, or from about 100 nm to about 1000 nm can be formed and maintained. The polymer matrix/agglomeration can be dissolved completely in both hot and cold aqueous solutions. Once dissolved the encapsulated cannabinoids will be released and suspended in the solution. The bulk particle size of the encapsulated cannabinoids can be maintained within a range from about 50 nm to about 20 microns once released from the polymer matrix/agglomeration.

The particle size of the milled powder can be between about 0.1 mm and about 5 mm. At this range the powder dissolves quickly in aqueous solutions. At particle sizes smaller than 0.1 mm, the powder begins to clump and prevents water penetration and at particle sizes above 5 mm diffusion into the matrix is slow and the powder takes longer to dissolve.

Additives

Additives can be included in the emulsion system or the polymer matrix to improve the flavor of the system. These flavoring additives include but are not limited to: sugar, sucrose, sorbitol, sucralose, saccharin sodium, sodium cyclamate, aspartame, neotame, acesulfame potassium, stevioside, sodium chloride, D-limonene, citric acid, essential oils, natural and artificial flavors.

According to alternate embodiments, it is possible to include functional or nutritional ingredients. Examples of such ingredients include, but are not limited to: caffeine, chamomile, b-vitamins, protein, omega-3 fatty acids, creatine, L-arginine, st. johns wort, horny goat weed, valerian root, phenylalanine, guarana, taurine, melatonin, turmeric, vitamins (A, B, E, C, etc.), echinacea, minerals (calcium, sodium, magnesium, zinc etc.), etc. Penetration enhancers such as alcohol, propylene glycol, polyethylene glycol, amino acids, peptides, proteins, polysorbates, dimethylsulfoxide, azones, terpenes, pyrrolidone, etc.

Yet other embodiments include a drug or therapy that has a pharmacological effect in the formulation. Examples of such additives include, but are not limited to: opioids, steroids, chemotherapeutic agents, immunosuppressive agents, immunostimulatory, anti pyretic, cytokines, cytotoxic agents, nucleolytic compounds, radioactive isotopes, enzymes, antibiotics, growth factors, protease inhibitors, analgesics, antidepressants, stimulants, antibodies, beta-blockers, anti-inflammatory agents, central nervous system depressants, etc.

Water Removal/Powder Drying:

The mixture of aqueous polymer and emulsion can be fully, partially, or substantially dehydrated, resulting in a dry solid mass. Dehydration can completely remove water, maintain the dispersed and substantially homogenous distribution of encapsulated cannabinoids, and maintains particle sizes for encapsulated cannabinoids between 50 nm and 20 microns. Dehydration can also achieve encapsulation efficiencies above 70%, or above 80% or above 95%, and result in a dry solid that can be milled into a free flowing powder with 500-3000 micron particles. The resulting powder can rapidly be dissolved in aqueous liquids and release the encapsulated cannabinoids.

Lyophilization is an example of such a dehydration method. It is capable of dehydrating the system at temperatures below 100° C. using sub-atmospheric pressures. Dehydrating below 100° C. preserves the entrapment and encapsulation of volatile compounds such as terpenes that would otherwise begin to boil at temperatures of about 120° C. at 1 atm. Lyophilization is also key to controlling the dissolution properties of the resulting powder. The pore size, porosity, and polymer composition dictate how quickly the powder will dissolve in both hot and cold aqueous liquids. The polymer composition is described above and the pore size and porosity are controlled by the initial water content and the lyophilization conditions. The initial water content of the system can be between 10% water by weight and 99% water by weight. Lyophilization can occur between 200 mTorr and atmospheric pressure and temperatures between −80° C. and 100° C., until the system is completely dehydrated. The resulting pore sizes are between 100 nm and 1 mm, with overall porosities of 10% to 90% of interconnected void space. This results in a powder that is quickly hydrated and self emulsifies. The resulting powder with dissolve on time scales between 10 seconds and 5 minutes

Freeze drying (lyophilization) can also prevent aggregation of particles, maintain particle sizes, has a high (optionally the highest) encapsulation efficiency and results in rapid dissolution of the powder in aqueous liquids. Encapsulated cannabinoids embedded in maltodextrin and freeze dried maintain particle sizes of 50 nm to 2 microns and an encapsulation efficiency above 90%. Maltodextrin with high DE values, at high concentrations, improves the stability of particles during the freezing process. Flash freezing in liquid nitrogen can be performed prior to freeze drying. The combination of maltodextrin and flash freezing results in inconsequential ice crystal formation that may cause destabilization of the matrix. The present embodiment prevents or at least mitigates particle aggregation, maintains particle sizes of 50 nm to 20 microns, and prevents irreversible agglomeration of maltodextrin. The resulting powder has improved aqueous dissolution and dissolves <5 min at 4° C.

Other methods of dehydrating include but are not limited to: spray drying, shell freezing, convection oven, evaporation, microwaving, rotary evaporation, and vacuum drying. Other than freeze drying, spray drying has the potential for maintaining small particle size and a high encapsulation efficiency. It is also capable of making powders with small particle sizes, resulting in powder particles that are less than 1 micron. This is advantageous for applications that require fine powder particle sizes such as: fast dissolving powders, nebulizers/inhalers, intravenous injections, etc. Another effective method for dehydrating the emulsion/maltodextrin system is microwave oven. It has the advantage of removing all of the water content in a matter of minutes.

Applications

Sublingual and buccal dosage forms require penetration of the drug particle through the epithelial layer and the mucosal membranes. In this fashion the drug is capable of being directly introduced into the bloodstream. It bypasses the GI tract and first pass effect in the liver, resulting in fast first onset of action, e.g., less than 30 minutes, and a high bioavailability, 2-5 fold increase compared to other dosage forms. Drug loaded particles can be smaller than 200-400 nm to effectively penetrate through tissues under the tongue and in the cheek. The present emulsions with particle sizes smaller than 400 nm can rapidly be absorbed sublingually, resulting in 5-10 min for the first onset of action and 2-5 fold increase bioavailability compared to a conventional cannabis oil in carrier oil systems. The present emulsion can be converted into a powdered dosage form that can easily be reconstituted into an aqueous solution that results in drug loaded particles less than 400 nm that can be rapidly absorbed sublingually with first onset of action times of 5-10 min and a 2-5 fold increased bioavailability compared to cannabinoids neat or dissolved in edibles oils.

Examples cannabinoid powder sublingual/buccal:

Applied directly as a powder under the tongue or in the cheek. The powder can also be put in a pouch sachet and placed in the mouth. The powder can be used for sublingual or buccal absorption by incorporation in a pouch, film, gel, lozenge, dissolvable strip, muco-adhesive, or any sublingual/buccal delivery system that allows transport of the cannabinoid particles into the mucosa tissues. The powder can be combined with a variety of excipient media as long as it does not change the sublingual/buccal absorption properties of the powder.

Powder Formulas: described above

Cannabinoid concentration: 1-200 mg/g cannabinoids (THC, CBD, etc.)

Dissolving time: 30 seconds to 5 minutes

Cannabinoid particle size after dissolution: 50 nm to 2 microns, or between 100-400 nm

First onset of action: 5-20 minutes

Flavor profile: neutral

Beverages and liquid oral dosage forms made using emulsions have improved stability when the particle size is reduced. Beverages and liquid oral dosage forms made with the cannabinoid emulsions of this invention, having particle sizes smaller than 2000 nm have a long term stability of at least 3 months. These emulsions are stable regardless of the beverage composition, pH, and temperature. The cannabinoid emulsions, with cannabinoid particles sizes of 50 nm to 2 microns, can be converted into a free flowing powder. This powder can then be reconstituted into a beverage or liquid oral dosage form. The powder is self emulsifying once hydrated, quickly and easily dispersing the encapsulated cannabinoids homogeneously throughout the entirety of the beverage. The beverage, or oral dosage form, will be stable from creaming, sedimentation, flocculation, phase separation, coalescence for a minimum of 24 hours, and optionally for 1-6 months. The reconstituted encapsulated drug has average or bulk particle sizes of no more than 1000 nm and optionally less than 500 nm.

Once reconstituted into a beverage. Beverages made using either the original emulsion or a powdered version of the emulsion will exhibit a rapid first action of onset, less than 30 min, with increased bioavailability, a 2-5 fold increased bioavailability compared to conventional cannabinoids dissolved in carrier oils. The encapsulated cannabinoid powder does not alter the flavor, texture or aroma of the beverage or oral dosage form. This is accomplished by choosing the appropriate cannabis oils, correct emulsifiers and any flavor modifiers that can aid with masking unwanted flavor notes. The cannabinoid powder herein can be flavor neutral due to encapsulation and may not influence the flavors of beverages and oral dosage forms. The powder can be used to make RTD beverages, powdered drink mixes, health drinks, juices, protein drinks, coffee (brewed or from grinds), tea (brewed or from leaves), beer, wine, cocktails, kombucha, elixirs, milk (regular, soy, almond, etc.) soft drinks and sodas, infused water, sparkling water. The powder both hydrated and dry is stable when combined with a variety of beverage ingredients including but not limited to: acids, bases, oils, carbonation, sugars, salts, electrolytes, waxes, solvents, sweeteners, proteins, fats, flavoring, coloring, etc. The hydrated powder is stable across a wide range of beverage processing methods including but not limited to: pasteurization, chilling, homogenization, mixing, heating (100° C.), high pressure processing and sterilization, filtration, bottling, carbonation, etc. Examples beverages made using cannabinoid emulsions or powdered encapsulated cannabinoids include, dissolved cannabinoid powder in a beverage:

1. Water: 0.04-40 mg/ml THC, stable 1 month+; flavor minimal; effects 15-30 min

2. Coffee: 0.04-40 mg/ml THC, stable 1 month+: flavor neutral; effects 15-30 min

3. Cranberry Juice: 0.04-40 mg/ml, stable 1 month+; flavor neutral; effects 15-30 min

4. Cold pressed juice (orange, carrot, pineapple): 0.04-40 mg/ml, stable 1 month+; stable after high pressure sterilization, flavor neutral; effects 15-30 min

5. Electrolyte Drink (Gatorade): 0.04-40 mg/ml, stable 1 month+; flavor neutral; effects 15-30 min

6. Ultra pasteurized milk: 0.04-40 mg/ml, stable 1 month+; flavor neutral; effects 15-30 min

Edible dosage forms can be made with the encapsulated cannabinoid powder. The encapsulation of the cannabinoids prevents the influence of cannabinoids/off flavors in the taste of the edibles. The powdered form easily mixes and evenly disperses into solid particulates, molten ingredients and liquids. In this regard the powdered cannabinoids can create an edible with a homogenous distribution of cannabinoids throughout. Edibles made using encapsulated cannabinoids will exhibit a reduced time for the first onset of action, less than 30 min; compared to 1 hour on average. Additionally edibles made using the powdered encapsulated cannabinoids reach peak blood concentrations in less than 2 hours compared to edibles made using cannabinoid rich edible oils that take 4-6 hours to reach peak blood concentrations. The encapsulated cannabinoid powder can be used in a wide variety of edible products including: baked goods, chocolates, candies, pills/capsules, edible oils, honey, agave, sweeteners, sugars, mints, lozenges, seasonings. The powder is stable with a wide range of ingredients commonly used to make edibles, this includes but is not limited to: acids, bases, oils, sugars, salts, proteins, fats, electrolytes, waxes, solvents, sweeteners, flavoring, coloring, etc. The powder is stable over a wide range of processes used to make edibles, processes include but are not limited to: baking/cooking (300-500° F.), mixing, homogenizing, dehydrating, dissolving, mechanical deformation, milling/grinding, pasteurization, high pressure sterilization. Examples edibles formulated with encapsulated cannabis powder:

1. Cookies: 0.5-20 mg/g cannabinoids, Baked (350° F. 25 min), Homogeneously blended in, stable 4+months, flavor neutral, effects in less than 30 minutes.

2. Chocolate: 0.5-70 mg/g cannabinoids, melted and tempered (90° F.), Flavor neutral, effects in less than 30 minutes. Mixed in, does not separate and creates a homogeneous mixture.

3. Honey: 0.5-20 mg/g cannabinoids, mixed in easily and does not phase separate. The effects are felt in less than 30 minutes.

It is also possible to create oral dosage forms using the cannabinoid powder. Similar to edible dosage forms, oral dosage forms made using the encapsulated powder have reduced onset times and higher bioavailability. However, it is possible to create oral dosage forms with alternative functionality. These alternative functionalities include: time release, instant release, enteric coatings and site specific adsorption. Examples of oral dosage forms made with encapsulated cannabis powder:

Capsules: 0.5-100 mg per capsule, first onset of action less than 30 minutes. Contained in gelatin capsules. The capsule can also be made with delayed release or extended release excipients that prolong the drug exposure.

Pressed tablets: 0.5-100 mg per tablet, first onset of action less than 30 minutes. Pressed using any necessary excipients and binders including but not limited to: microcrystalline cellulose, hypromellose (HPMC), Eudagrit. The tablet can also be made with delayed release or extended release excipients that prolong the drug exposure.

Topical dosage forms can be made with the encapsulated cannabinoid powder. The polymer matrix/agglomeration is to be dissolved in an aqueous solution to liberate the encapsulated cannabinoids. This can be done prior to formulation of the topical or can occur naturally in an aqueous based topical. The liberated encapsulated cannabinoids can be of sufficiently small size to penetrate the epidermis and create an effective treatment. For example, the encapsulated cannabinoids can have a particle size of less than 1000 nm once dissociated from the polymer matrix/agglomeration, or less than 500 nm, or less than 300 nm. Topical dosage forms include: patches, gels, ointments, creams, balms, lotions, salves, chapstick, soap, moisturizer, conditioner, shampoo, cosmetics, etc.

Other possible dosage forms include but are not limited to: pulmonary inhalation (nebulizer), rectal/vaginal suppository, intravenous injection, intranasal spray, ophthalmological drops, and intramuscular injection. The encapsulated cannabinoid powder must be combined with the proper excipients and solutions to make effective therapies for these alternative dosage forms.

In this application, the use of the singular includes the plural unless specifically stated otherwise. As used herein, the singular forms “a,” “an” and “the” are intended to include the plural forms as well, unless the context clearly indicates otherwise.

As used herein, certain values can be expressed using a term of approximation, such as “about” or “approximately.” Such terms are intended to also include the exact amount. For example, “about X percent” or “approximately X percent” exactly X percent, as well as values within typical experimental error of X for the application or purpose intended.

Throughout this disclosure, all parts and percentages are by weight (wt % or mass % based on the total weight) and all temperatures are in ° C. unless otherwise indicated.

In this application, the use of “or” means “and/or” unless stated otherwise. It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method of forming a particulate material derived from a cannabis plant, the method comprising:
introducing a component comprising at least one of: (i) a cannabinoid, and (ii) a terpene, to a polymer to produce a polymeric mixture;
dispersing the component in the polymeric mixture;
at least partially dehydrating the polymeric mixture to encapsulate the component within a polymeric material derived from the polymer, wherein the polymeric material is water soluble; and
processing the dehydrated polymeric mixture to form particulates comprising the component encapsulated within shells formed from the polymeric material.

2. The method of claim 1, wherein the component comprises a cannabinoid selected from a group consisting of:
delta-9-tetrahydrocannabinolic acid,
delta-9-tetrahydrocannabinol,
cannabidiol acid,
cannabidiol,
cannabinol,
cannabigerol,
cannabichromene,
tetrahydrocannabivarin, and
cannabidivarin.

3. The method of claim 1, wherein the component comprises a combination of both a cannabinoid and a terpene.

4. The method of claim 1, wherein the component comprises a cannabinoid, and is devoid of a terpene.

5. The method of claim 1, wherein the component comprises at least 25% of the at least one of the cannabinoid and the cannabis-based terpene, by weight.

6. The method of claim 1, wherein the component comprises at least 50% of the at least one of the cannabinoid and the cannabis-based terpene, by weight.

7. The method of claim 1, wherein the component comprises at least 65% of the at least one of the cannabinoid and the cannabis-based terpene, by weight.

8. The method of claim 1, wherein the polymeric material is approved for human consumption by a government regulatory body.

9. The method of claim 1 further comprising introducing a triglyceride to the component, the polymer, and/or the polymeric mixture.

10. The method of claim 9, wherein the triglyceride comprises a medium chain triglyceride comprising an aliphatic tail that is between six (6) and twelve (12) carbon atoms in length.

11. The method of claim 9, wherein the triglyceride comprises a medium chain triglyceride selected from a group consisting of:
caproic acid,
caprylic acid,
olive oil,
vegetable oil,
capric acid,
lauric acid, and
fractionated coconut oil.

12. The method of claim 9, wherein introducing the triglyceride comprises introducing a suitable quantity of the triglyceride to establish a ratio of a weight of the component to a weight of the triglyceride in the polymeric mixture that is at least 1:1.

13. The method of claim 12, wherein the suitable quantity of the triglyceride establishes a ratio of the weight of the component to the weight of the triglyceride in the polymeric mixture that is less than 1:10.

14. The method of claim 1, wherein processing the dehydrated polymeric mixture comprises mechanically machining the dehydrated polymeric mixture to create a powder having an average particle size for a majority of the particulates that is no greater than five (5) mm.

15. The method of claim 14, wherein the average particle size is between one tenth (0.1) mm and three (3) mm.

16. The method of claim 1, wherein dehydrating the polymeric mixture comprises lyophilization of the polymeric mixture under a vacuum at a sub-atmospheric pressure and at an operational temperature of less than 100° C.

17. The method of claim 1, wherein dehydrating the polymeric mixture comprises spray drying the polymeric mixture.

18. The method of claim 1 further comprising introducing a polysaccharide or a protein as an additive to the component, the polymer, or the polymeric mixture.

19. The method of claim 1, wherein introducing the component to the polymer is performed in an absence of a polysaccharide.

20. The method of claim 1, wherein the component is in a fluid or gel state, and encapsulating the component comprises:
inputting a signal to the polymeric mixture to form droplets of the component suspended in the polymeric mixture before completing dehydration.

21. The method of claim 1, wherein the component is a liquid, or the component is a gel having a viscosity between 0.3 centipoise and 250,000 centipoise.

22. The method of claim 1, wherein most dehydration of the polymeric mixture occurs at temperatures below 180° F.
